(19) United States
(12) Patent Application Publication
(10) Pub. No.: US 2024/0189467 A1
(43) Pub. Date: Jun. 13, 2024

(54) Disinfecting Medical Cart Cover

(71) Applicant: Bard Access Systems, Inc., Salt Lake City, UT (US)

(72) Inventors: Robin Scott Urry, Syracuse, UT (US); Debra K. Cloward, Springville, UT (US); Dustin Payne, West Jordan, UT (US); Matthew J. Prince, Herriman, UT (US); Craig Hayden, Sandy, UT (US); Timothy M. Snelling, Salt Lake City, UT (US)

(21) Appl. No.: 18/077,994

(22) Filed: Dec. 8, 2022

(51) Int. Cl. A61L 2/10; A61L 2/24

(52) U.S. Cl. CPC A61L 2/10; A61L 2/24; A61L 2202/16; A61L 2202/24

(57) ABSTRACT

A system and method for disinfecting medical devices includes a covering for a medical cart that exposes medical devices disposed on the cart to a disinfecting ultraviolet light. The covering includes a light emitting system that includes a number of UV light sources, such as LEDs, configured to define an ultraviolet light environment beneath the covering. The light emitting system may include one or more light pipes and/or optical fibers. systems and methods. An opaque layer of the covering contains the ultraviolet light environment beneath the covering. Portions of the light emitting system suspend from the covering so as to emit UV light between adjacent medical devices on the cart.

### BACKGROUND

Multi-use medical devices are generally sterilized between uses via a facility sterilizing system, such as an autoclave. Once sterilized, the multi-use medical devices start becoming less sterile upon removal from the autoclave. In typical instances, sterile coverings (e.g., blankets or towels) may be placed over the medical devices to help maintain the sterility of the medical devices. However, since the general environment (e.g., hospital rooms) of the healthcare facility is non-sterile, the medical devices may become less sterile over time presenting an infection risk to the patient. Furthermore loss of sterility is generally not detectable.

Disclosed herein are systems and methods for disinfecting medical devices that address the forgoing.

### SUMMARY

Disclosed herein is a disinfection system that, according to some embodiments, includes a covering configured for placement over a medical cart, where the covering includes a light emitting system configured to define an ultraviolet light environment beneath the covering such that medical devices included with the cart are disinfected by ultraviolet light. In some embodiments, the covering is configured to surround the cart. In some embodiments, the light emitting system includes a battery power source.

In some embodiments, the covering further includes an opaque layer coupled with the light emitting system, where the opaque layer is configured to contain the ultraviolet light beneath the covering.

In some embodiments, the covering further comprises a fabric panel configured to provide a mechanical structure to the covering, and the light emitting system is coupled with the fabric panel. In some embodiments, the light emitting system is integrated into the fabric panel.

In some embodiments, the light emitting system includes a plurality of light emitting diodes configured to project the ultraviolet light beneath the covering, and in some embodiments, at least a subset of the plurality of light emitting diodes suspend a distance away from the covering to extend a depth of the ultraviolet light environment.

In some embodiments, the light emitting system includes a passive light projector extending across the covering, where the passive light projector is optically coupled with an ultraviolet light source. In some embodiments, the ultraviolet light source includes a light emitting diode.

In some embodiments, the passive light projector includes at least one of (i) a light pipe configured to project the ultraviolet light away from the underside, or (ii) an optical fiber having a plurality of fiber optic gratings configured to project the ultraviolet light away from the optical fiber, and in some embodiments, at least a portion of the passive light projector extends a distance away from the covering to extend a depth of the ultraviolet light environment.

In some embodiments, the system further includes an ultraviolet light source optically coupled with the passive light projector. In some embodiments, the ultraviolet light source is detachably coupled with the passive light projector via an optical connector. In some embodiments, the ultraviolet light source is detachably coupled with the covering.

Also disclosed herein is a method for disinfecting medical devices that, according to some embodiments, includes (i) placing the medical devices on a cart, (ii) placing a covering over the cart, and (iii) defining an ultraviolet light environment beneath the covering.

In some embodiments of the method, the covering includes a light emitting system disposed on an underside of the covering, and defining the ultraviolet light environment includes activating an ultraviolet light source of the light emitting system.

In some embodiments of the method, the light emitting system includes one or more suspended portions extending away from the underside, and the method further includes inserting a suspended portion of the light emitting system between adjacent ones of the medical devices.

In some embodiments of the method, the light emitting system includes a passive light projector optically coupled with the ultraviolet light source, where the passive light projector includes at least one of a light pipe or an optical fiber.

In some embodiments, the method further includes automatically deactivating the ultraviolet light source after a defined activation time period.

These and other features of the concepts provided herein will become more apparent to those of skill in the art in view of the accompanying drawings and following description, which describe particular embodiments of such concepts in greater detail.

### DRAWINGS

FIG. 1A illustrates a medical device disinfecting system, according to some embodiments.

FIG. 1B is a detailed illustration of a covering of the medical device disinfecting system of FIG. 1A, according to some embodiments.

FIG. 2 is a cross-sectional side view of a portion of the covering of FIG. 1B, according to some embodiments.

FIG. 3 illustrates a first embodiment of a light emitting system of the covering of FIG. 1B, according to some embodiments.

FIG. 4 illustrates a second embodiment of a light emitting system of the covering of FIG. 1B, according to some embodiments.

FIG. 5 illustrates a third embodiment of a light emitting system of the covering of FIG. 1B, according to some embodiments.

FIG. 6 illustrates a flow chart of an exemplary method of disinfecting medical devices, according to some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, and optical interaction. Two components may be coupled with each other even though they are not in direct contact with each other. For example, two components may be coupled with each other through an intermediate component.

Unless defined otherwise, all technical and scientific terms used herein have the same meaning as commonly understood by those of ordinary skill in the art.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

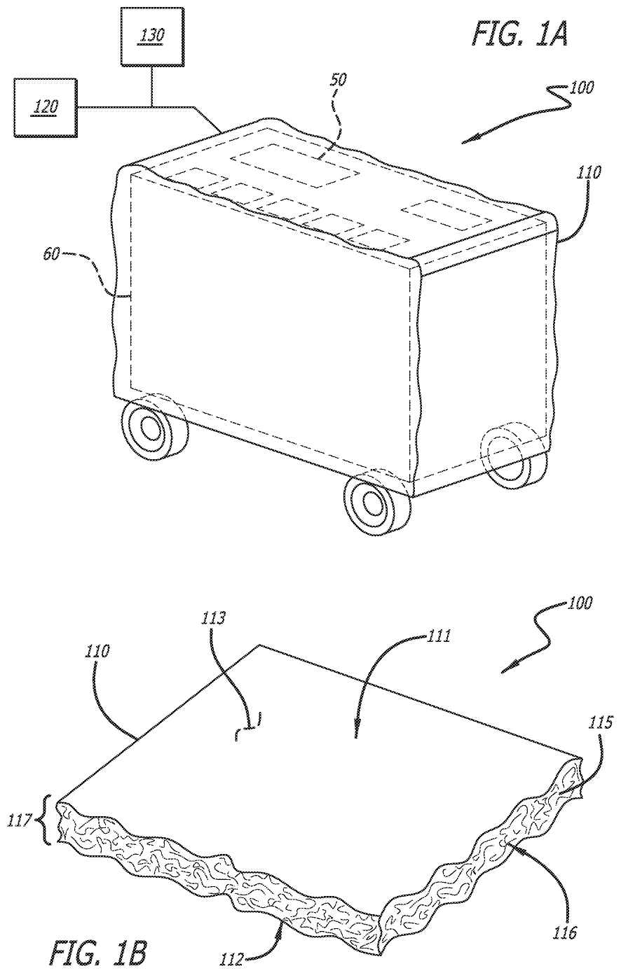

FIG. 1A illustrates a disinfecting system (system) 100 configured for disinfecting multi-use medical devices in a healthcare environment, such as a hospital, for example. The system 100 generally includes a covering 110 configured for placement over a medical cart (cart) 60, where the cart 60 includes a number of medical devices 50 placed thereon. The covering 110 is generally configured for placement onto and removal from the cart 60 by a clinician. For example, during use, the clinician may place the medical devices 50 on the cart 60 and cover the cart 60 including the medical devices 50 with the covering 110. The clinician may move the cart 60 to a location of use (e.g., an operating room) after which the clinician may remove the covering 110 from the cart 60 exposing the medical devices 60.

The system 100 may be generally disinfect the medical devices 50 or assist in maintaining a sterile condition of the medical devices 50 after sterilization via a separate sterilizing system, such as an autoclave, for example. In some instances, multi-use medical devices start becoming less sterile upon removal of from the autoclave. In typical instances, sterile coverings (e.g., blankets or towels) are placed over the medical devices to help maintain the sterility of the medical devices. However, since the general environment (e.g., hospital rooms) of the healthcare facility is non-sterile, the medical devices may become less sterile over time presenting an infection risk to the patient. As such, in such instances, the system 100 may be configured to re-disinfect (e.g., re-sterilize) portions of the medical devices that are prone to contamination between initial sterilization and use.

The medical devices 50 may be placed on a top surface of the medical cart 60 or otherwise coupled with the medical cart 60. The covering 110 may generally take the form of a fabric covering such as a blanket or sheet, for example. The covering 110 may be configured to drape over the top surface of the cart 60 and extend down the sides of the cart 60, such that the covering 110 entirely surrounds the cart 60. In some embodiments, the covering 110 may include a pre-formed shape consistent with the cart 60, i.e., the covering 110 may include pre-fit corners consistent with the shape of the cart 60. The covering 110 may also be configured to be folded into a storage configuration, thereby facilitating placement on a shelf, for example. In some embodiments, the covering 110 may be configured to undergo a sterilization process, such as via radiation, autoclaving, or chemical exposure.

The covering 110 is configured to disinfect the medical devices 50 via exposure of the medical devices 50 to ultraviolet light emanating from the covering 110. The system 110 includes a power source 120 coupled with the covering 110, where the power source 120 may include a battery. An activation device 130 is configured to activate and/or deactivate a disinfection process as described in further detail below.

FIG. 1B is a perspective view of the covering 110 removed from the cart 60. For illustrative purposes, the covering 110 is shown in a flat shape. The covering 110 generally defines a top side 111, an underside 112, and an area 113. In an activated state, the covering 110 defines an ultraviolet light environment 115 extending (i) across the area 113 and (ii) away from the underside 112 a depth distance 117. The ultraviolet light environment 115 includes an ultraviolet light 116 configured to disinfect surfaces (e.g., external surfaces of the medical devices 50) disposed within the ultraviolet light environment 115. In some embodiments, the ultraviolet light 116 may include wavelengths between about 100 nm and 280 nm.

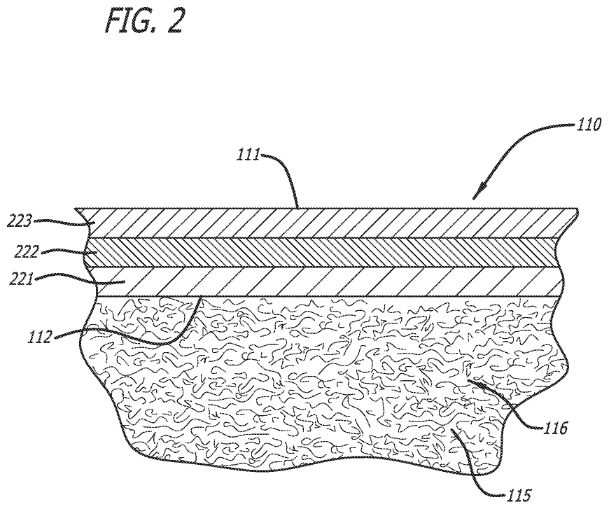

FIG. 2 illustrates a cross-section side view of a portion of the covering 110, according to some embodiments. The covering 110 may include a number of layers extending across the area 113 (FIG. 1B). The covering includes a light emitting layer 221 configured to define the ultraviolet light environment 115 when activated via the power source 120. The light emitting layer 221 includes light emitting devices that project ultraviolet light throughout the ultraviolet light environment 115 as further described below.

The covering 110 may also include a fabric panel 222. The fabric panel 222 may define the mechanical structure of the covering 110. The light emitting layer 221 is coupled with the fabric panel 222. In some embodiments, the light emitting layer 221 is coupled with an underside of the fabric panel 222. In some embodiments, the light emitting layer 221 may be integrated into the fabric panel 222. The fabric panel 222 may be formed of a panel material suitable for use within the healthcare environment. In some embodiments, the panel material may be a non-sheading material. The panel material may be sterilizable, i.e., configured to withstand multiple sterilizing procedures.

The covering 110 may also include an opaque layer 223. The opaque layer 223 is configured to prevent or inhibit ultraviolet light from projecting away from the top side 111 of the covering 110. Said another way, the opaque layer 223 is the configured to contain the ultraviolet light 116 to the underside 112 of the covering 110. In some embodiments, the opaque layer 223 is coupled with a topside of the fabric panel 222. In some embodiments, the opaque layer 223 may be integrated into the fabric panel 222, i.e., the fabric layer 222 may be configured to contain the ultraviolet light 116 to the underside 112 of the covering 110.

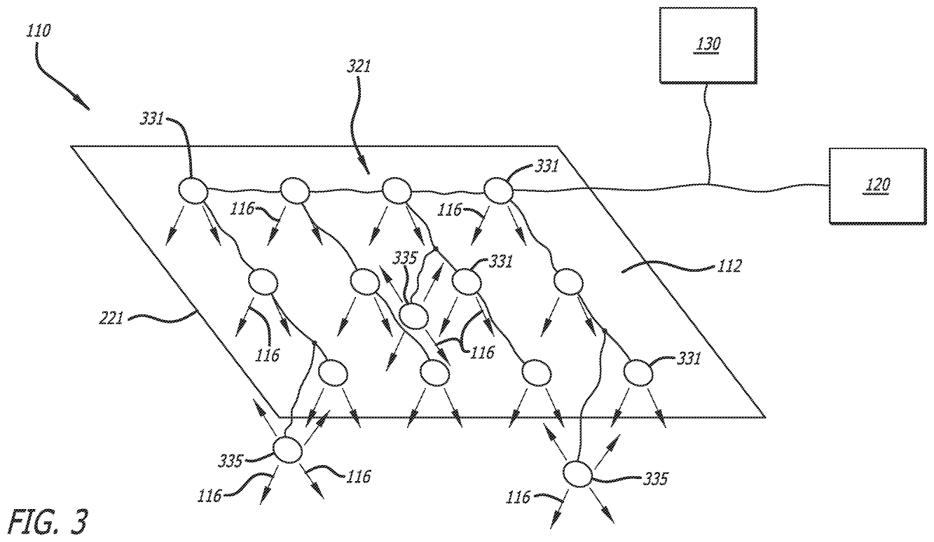

FIG. 3 is a perspective underside view of the covering 110 illustrating a first embodiment of a light emitting system of the light emitting layer 221. The light emitting system 321 includes a plurality of individual ultraviolet (UV) light sources 331. The UV sources 331 may be attached to the fabric layer 222 (FIG. 2). The UV light sources 331 are configured to project UV light 116 downward in a dispersed fashion to define the ultraviolet light environment 115 (FIGS. 1A-1B). In some embodiments, the UV light sources 331 may include light emitting diodes (LEDs). The UV light sources 331 may be arranged in an array across the underside 112 of the covering 110. The UV light sources 331 are electrically connected to the power source 120.

In some embodiments, the activation device 130 may include a programmable timer so that the clinician may define an activation schedule. In some embodiments, the programmable timer may enable to activation device 130 to activate the UV light sources 331 according to a defined schedule, such as at the beginning of each day, or after a defined time period since the last activation, for example. In some embodiments, the programmable timer may enable the clinician to define an activation time period (i.e., an “on” duration for the UV light sources 331). As such, the activation device 130 may automatically deactivate the UV light sources 331 after the defined activation time period.

In some embodiments, a subset of the UV light sources 331 may include suspended UV light sources 335 (e.g., LEDs) that suspend a distance from the fabric layer 222 so as to extend the depth of the ultraviolet light environment 115, i.e., increase the depth distance 117. The suspended UV light sources 335 may be configured to project UV light 116 in multiple directions, such as downward, upward, and laterally outward, for example, away from the UV light source 335. In some instances, the suspended UV light sources 335 be disposed amongst (including between) medical devices 50. By projecting UV light 116 in multiple directions, the suspended UV light sources 335 may project the UV light 116 onto an increased surface area of the medical devices 50 in relation to the UV light sources 331, thereby enhancing disinfection of the medical devices 50.

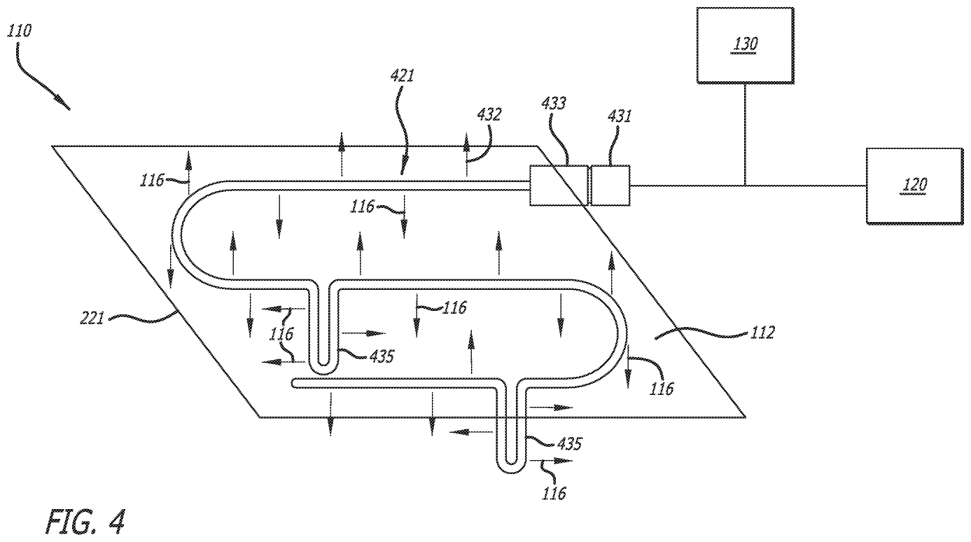

FIG. 4 is a perspective underside view of the covering 110 illustrating a second embodiment of the light emitting system of the light emitting layer 221. The light emitting system 421 includes a UV light source 431 optically coupled with a light pipe 432 (i.e., a passive light projector) via an optical connector 443. The UV light source 431 is coupled to the power source 120 via the actuating device 130. The optical connector 443 allows for disconnection of the UV light source 431 from the light pipe 432 so that the UV light source 431 including the power source 120 and the actuating device 130 may be separated from the covering 110.

The light pipe 432 is configured to (i) propagate UV light 116 along the length of the light pipe 432 and (ii) project the UV light 116 laterally and radially away from the light pipe 432 to define the ultraviolet light environment 115. The light pipe 432 may be attached to the fabric layer 222. The light pipe 432 may include any number of straight and curved sections so as to effectively extend across an entire area of the underside 112.

In some embodiments, the light pipe 432 may include a number of the suspended sections 435 that suspend a distance from the fabric layer 222 so as to extend the depth of the ultraviolet light environment 115, i.e., increase the depth distance 117. In some instances, the suspended sections 435 be disposed amongst (including between) medical devices 50. As such, the suspended sections 435 may project the UV light 116 onto an increased surface area of the medical devices 50 in relation to the light pipe 432 generally, thereby enhancing disinfection of the medical devices 50.

Although the illustrated embodiment of the light emitting system 421 includes a single light pipe 432 coupled with a single light UV light source 431, in other embodiments, the light emitting system 421 may include any number of the light pipes 432 and/or UV light sources 431 coupled together in any suitable fashion.

As the activation device 130 may include a programmable timer as discussed above, the activation device 130 may (i) activate the UV light source 431 according to a defined schedule, and/or (ii) automatically deactivate the UV light source 431 after the defined activation time period.

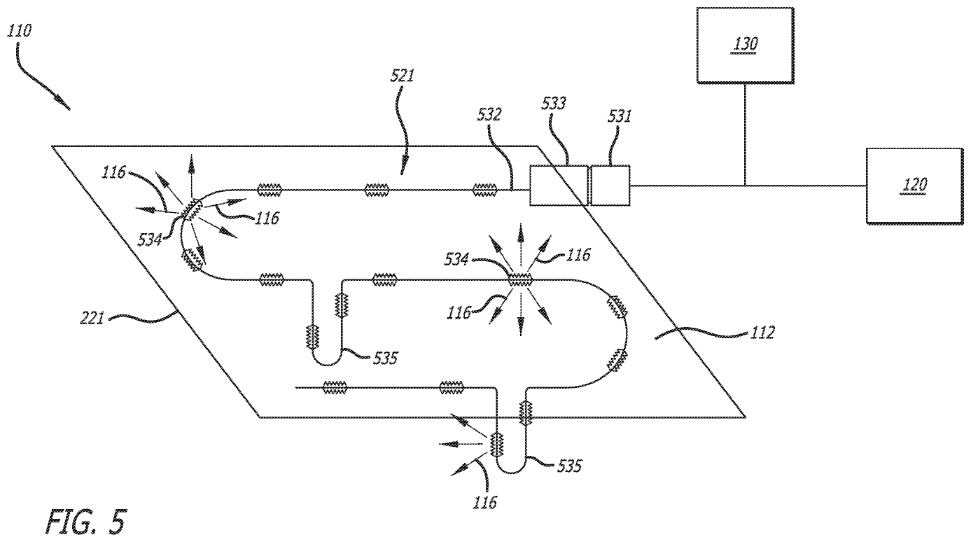

FIG. 5 is a perspective underside view of the covering 110 illustrating a third embodiment of the light emitting system of the light emitting layer 221. The light emitting system 521 includes a UV light source 531 optically coupled with an optical fiber 532 (i.e., a passive light projector) via an optical connector 543. The UV light source 531 is coupled to the power source 120 via the actuating device 130. The optical connector 543 allows for disconnection of the UV light source 531 from the optical fiber 532 so that the UV light source 531 including the power source 120 and the actuating device 130 may be separated from the covering 110.

The optical fiber 532 is configured to propagate UV light 116 along the length of the optical fiber 532. The optical fiber 532 includes a plurality of gratings 535 disposed along the optical fiber 532 where each grating 535 is configured to project the UV light 116 laterally away from the optical fiber 532 in a dispersed fashion to define the ultraviolet light environment 115. The optical fiber 532 may be attached to the fabric layer 222. The optical fiber 532 may include any number of straight and curved sections so as to effectively extend across an entire area of the underside 112.

In some embodiments, the optical fiber 532 may include a number of the suspended sections 535 that suspend a distance from the fabric layer 222 so as to extend the depth of the ultraviolet light environment 115, i.e., increase the depth distance 117. In some instances, the suspended sections 535 be disposed amongst (including between) medical devices 50. As such, the suspended sections 535 may project the UV light 116 onto an increased surface area of the medical devices 50 in relation to the optical fiber 532 generally, thereby enhancing disinfection of the medical devices 50.

Although the illustrated embodiment of the light emitting system 521 includes a single optical fiber 532 coupled with a single light UV light source 531, in other embodiments, the light emitting system 521 may include any number of the optical fibers 532 and/or UV light sources 531 coupled together in any suitable fashion.

As the activation device 130 may include a programmable timer as discussed above, the activation device 130 may (i) activate the UV light source 431 according to a defined schedule, and/or (ii) automatically deactivate the UV light source 431 after the defined activation time period.

The system 100 may include any of the light emitting systems 321, 421 or 521 individually. In some embodiments, the system 100 may include any combination of the light emitting systems 321, 421, and 521.

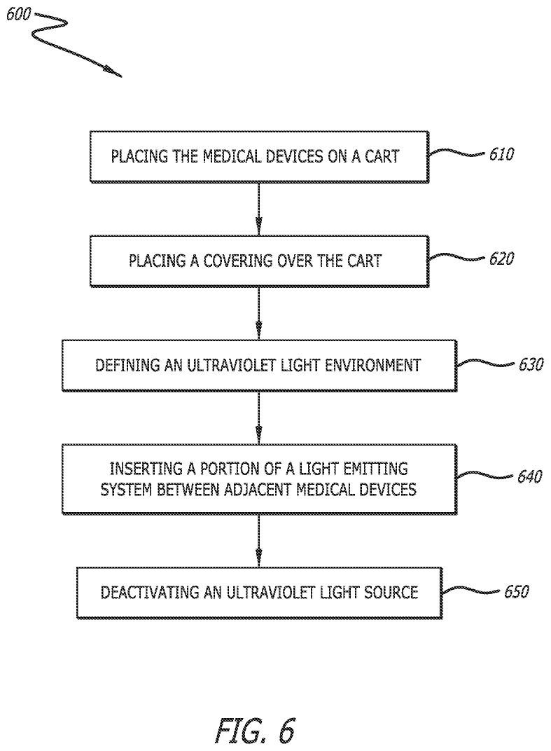

FIG. 6 illustrates a flow chart of an exemplary method 600 for disinfecting a number of medical devices, where the method 600 includes all or any subset of the following steps, actions, or processes. The method 600 generally includes placing the medical devices on a cart (block 610) and placing a covering over the cart (block 620), where the covering includes a light emitting system configured to define an ultraviolet light environment beneath the covering. The method 600 further includes defining an ultraviolet light environment (block 630) beneath the covering. In some embodiments, defining an ultraviolet light environment includes activating an ultraviolet light source, where the ultraviolet light source is operatively coupled with the light emitting system of the covering.

In some embodiments of the method, the light emitting system includes suspended portions configured for disposition between adjacent medical devices on the cart. As such, the method 600 may include inserting a portion of the light emitting system between adjacent medical devices (block 640) on the cart.

In some embodiments of the method 600, the light emitting layer includes a passive light projector optically coupled with the ultraviolet light source, where the passive light projector includes at least one of a light pipe or an optical fiber. As such, the method 600 may include inserting one or more portions of the light pipe or an optical fiber between adjacent medical devices on the cart.

In some embodiments of the method 600, the covering includes an opaque layer coupled with the light emitting layer, where the opaque layer is configured to contain the ultraviolet light environment beneath the covering. As such, the method 600 may include containing the ultraviolet light environment beneath the covering.

The method 600 may further include deactivating the ultraviolet light source (block 650). In some embodiments, deactivating the ultraviolet light source includes automatically deactivating the ultraviolet light source after a defined activation time period.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A disinfection system, comprising:
a covering configured for placement over a medical cart, the covering comprising a light emitting system configured to define an ultraviolet light environment beneath the covering such that the cart and medical items thereon are disinfected by ultraviolet light.

2. The system according to claim 1, wherein the covering further comprises an opaque layer coupled with the light emitting system, the opaque layer configured to contain the ultraviolet light beneath the covering.

3. The system according to claim 1, wherein the covering is configured to surround the cart.

4. The system according to claim 1, wherein:
the covering further comprises a fabric panel configured to provide a mechanical structure to the covering, and
the light emitting system is coupled with the fabric panel.

5. The system according to claim 4, wherein the light emitting system is integrated into the fabric panel.

6. The system according to claim 1, wherein the light emitting system includes a plurality of light emitting diodes configured to project the ultraviolet light beneath the covering.

7. The system according to claim 6, wherein at least a subset of the plurality of light emitting diodes suspend a distance away from the covering to extend a depth of the ultraviolet light environment.

8. The system according to claim 1, wherein the light emitting system includes a passive light projector extending across the covering.

9. The system according to claim 8, further comprising an ultraviolet light source optically coupled with the passive light projector.

10. The system according to claim 8, wherein the ultraviolet light source includes a light emitting diode.

11. The system according to claim 8, wherein the passive light projector includes at least one of:
a light pipe configured to project the ultraviolet light away from the underside, or
an optical fiber having a plurality of fiber optic gratings configured to project the ultraviolet light away from the optical fiber.

12. The system according to claim 8, wherein at least a portion of the passive light projector extends a distance away from the covering to extend a depth of the ultraviolet light environment.

13. The system according to claim 9, wherein the ultraviolet light source is detachably coupled with the passive light projector via an optical connector.

14. The system according to claim 13, wherein the ultraviolet light source is detachably coupled with the covering.

15. The system according to claim 1, wherein the light emitting system includes a battery power source.

16. A method of disinfecting medical devices, comprising:
placing the medical devices on a cart;
placing a covering over the cart; and
defining an ultraviolet light environment beneath the covering.

17. The method according to claim 16, wherein:
the covering includes a light emitting system disposed on an underside of the covering, and
defining the ultraviolet light environment includes activating an ultraviolet light source of the light emitting system.

18. The method according to claim 17, wherein the light emitting system includes one or more suspended portions extending away from the underside, the method further comprising inserting a suspended portion of the light emitting system between adjacent ones of the medical devices.

19. The method according to claim 17, wherein:
the light emitting system includes a passive light projector optically coupled with the ultraviolet light source, and
the passive light projector includes at least one of a light pipe or an optical fiber.

20. The method according to claim 17, further comprising automatically deactivating the ultraviolet light source after a defined activation time period.
